(19) United States
(12) Patent Application Publication
(10) Pub. No.: US 2009/0292279 A1
(43) Pub. Date: Nov. 26, 2009

(54) Device and Method for Coordinated Insertion of a Plurality of Cryoprobes

(75) Inventors: Mordechai Bliweis, Haifa (IL); Roni Zvuloni, Haifa (IL)

Correspondence Address: MARTIN D. MOYNIHAN d/b/a PRTSI, INC., P.O. BOX 16446, ARLINGTON, VA, 22215, US

(73) Assignee: Galil Medical Ltd., Yokneam (IL)

(21) Appl. No.: 11/884,684

(22) PCT Filed: Jan. 25, 2007
(86) PCT No.: PCT/IL2007/000091

§ 371 Date: Jan. 21, 2009

Related U.S. Application Data
(60) Provisional application No. 60/762,110, filed on Jan. 26, 2006.

(30) Foreign Application Priority Data
Dec. 12, 2006 (US) 11637095
Dec. 18, 2006 (US) 11640309

(51) Int. Cl. A61B 18/02

(52) U.S. Cl. 606/21

(57) ABSTRACT

The present invention relates to devices and methods delivering a plurality of thermal ablation probes to an organic target in a body, the probes being delivered in a configuration and orientation enabling efficient and thorough ablation of a large target of complex shape. Preferred embodiments include introducers having individual probe channels shaped to direct inserted probes to diverge upon exiting the introducer, probes designed and constructed to bend in selected manner when exiting the introducer, and apparatus for stabilizing an introducer with respect to a target during insertion of treatment probes from introducer to target.

### RELATED APPLICATIONS

This Application claims the benefit of pending U.S. Provisional Patent Application No. 60/762,110 filed Jan. 26, 2006.

This Application is a continuation-in-part of pending U.S. patent application Ser. No. 11/640,309 filed Dec. 18, 2006, which is a continuation of U.S. patent application Ser. No. 10/660,478 filed Sep. 12, 2003, now U.S. Pat. No. 7,150,743, which is a continuation of U.S. patent application Ser. No. 09/860,486 filed May 21, 2001, now U.S. Pat. No. 6,760,037, which claims the benefit of U.S. Provisional Patent Application No. 60/242,455 filed Oct. 24, 2000, now expired.

This Application is also a continuation-in-part of pending U.S. patent application Ser. No. 11/637,095 filed Dec. 12, 2006, which is a continuation-in-part of U.S. patent application Ser. No. 10/660,478 filed Sep. 12, 2003, now U.S. Pat. No. 7,150,743, which is a continuation of U.S. patent application Ser. No. 09/860,486 filed May 21, 2001, now U.S. Pat. No. 6,706,037, which claims the benefit of U.S. Provisional Patent Application No. 60/242,455 filed Oct. 24, 2000, now expired.

Pending U.S. patent application Ser. No. 11/637,095 is also a continuation-in-part of pending U.S. patent application Ser. No. 11/055,597 filed Feb. 11, 2005, which is a continuation of U.S. patent application Ser. No. 09/987,689 filed Nov. 15, 2001, now abandoned, which is a continuation-in-part of U.S. patent application Ser. No. 09/860,486 filed May 21, 2001, now U.S. Pat. No. 6,706,037, which claims the benefit of U.S. Provisional Patent Application No. 60/242,455 filed Oct. 24, 2000, now expired.

Pending U.S. patent application Ser. No. 11/637,095 is also a continuation-in-part of pending U.S. patent application Ser. No. 11/185,699 filed Jul. 21, 2005, which is a divisional of U.S. patent application Ser. No. 10/151,310 filed May 21, 2002, now abandoned, which claims the benefit of U.S. Provisional Patent Application No. 60/300,097 filed Jun. 25, 2001, now expired, and U.S. Provisional Patent Application No. 60/291,990 filed May 21, 2001, now expired.

Pending U.S. patent application Ser. No. 11/637,095 also claims the benefit of pending U.S. Provisional Patent Application No. 60/762,110 filed Jan. 26, 2006.

Pending U.S. patent application Ser. No. 11/637,095 also claims the benefit of U.S. Provisional Patent Application No. 60/750,833 filed Dec. 16, 2005, now expired.

The contents of all the above-mentioned applications are incorporated herein by reference.

### FIELD AND BACKGROUND OF THE INVENTION

The present invention relates to devices and methods for thermal ablation of a surgical target within a body of a patient. More particularly, the present invention relates to use of an introducer for delivering thermal ablation probes to an organic target in a desired configuration and orientation.

Cryotherapy is often called upon to treat lesions which are larger than the size of the ice ball which can be formed by a single cryoprobe. Using, repositioning, and re-using a same probe to treat large lesion is impractical, given the time-consuming freezing, thawing, and re-freezing processes involved. Consequently, a plurality of probes is typically used to treat a large treatment target. Yet, it is difficult to accurately insert a plurality of cryoprobes into a body and to position those probes in such manner that their treatment heads are in desired target locations relative one to another and relative to a target lesion. The process is particularly difficult when long, thin cryoprobes are used. Yet treated organs are often deep within the body, and cryoprobes and associated sensor probes must penetrate thick layers of tissue to reach an intended treatment locus.

In prostate cryoablation, where insertion depth is relatively short, templates are used. U.S. Pat. No. 6,905,492 to Zvuloni et al. presents examples where templates are used to control cryoablation of large lesions using multiple cryoprobes. However, cryoprobes are typically only semi-rigid. Probes often bend or stray off course when inserted through thick layers of tissue. Once a cryoprobe has begun to be inserted into a body, a surgeon's ability to control the exact course of the probe's insertion is limited because of the probes inherent flexibility and because of the strength of internal tissue structures through which the probe must pass. In many contexts, for example in treatment of lesions within the abdomen, where probes must penetrate skin, fat, and muscular layers to reach a treatment target, use of templates to guide a plurality of probes to a target has been found to be unsatisfactory. In such contexts cryoprobes are often inserted individually, under endoscopic and/or ultrasound visual guidance. Such procedures, however, typically necessitate making a plurality of incisions to accommodate inserting a visual imaging (scope) apparatus, lighting instruments, cryoprobes, a manipulator for handling the probes, and means for inflating a body cavity to create a free visual field. Such a plurality of incisions is highly disadvantageous. Moreover, insertion of multiple probes is a time-consuming process. Since the patient is usually under general or at least local anesthesia, long operation times pose not only reduce productivity for the surgeon and healthcare facility, but increase patient discomfort and risks of complications.

An additional problem generally encountered during insertion of multiple treatment probes into a target organ is that treated organs are typically flexible and moveable, and do not maintain a fixed position during attempts by a surgeon to penetrate them with a plurality of cryoprobes or other needle-like treatment tools. Thus, in many cases it is important to hold and stabilize a body organ or other treatment target, to prevent its motion during insertion and positioning of treatment tools such as cryoprobes.

U.S. Pat. No. 6,494,844 to Van Bladel et al. discloses a cannula adapted to apply suction through a lumen of a catheter to a tumor or lesion. Val Bladel's lumen has a self-sealing valve through which a cryoprobe may be inserted while suction is applied. Van Bladel discloses a multiple coring needle. U.S. Pat. No. 6,551,255 to Van Bladel et al. discloses an adhesion probe for securing the tumor. The probe secures the tumor by piercing the tumor and providing a coolant to the distal tip to cool the tip, thereby causing adhesion between tip and tissue.

Cryoprobes are typically designed and constructed to produce very cold temperatures at their treatment tips. However, it is a well-known disadvantage of cryoprobes that cold cryogen exhausting from a treatment tip and flowing through a proximal shaft of the cryoprobe may cool that shaft to the point where the unintentionally cooled shaft causes undesired freezing damage to healthy untreated tissue proximate to the shaft. Thermal insulation layers are often provided surrounding cryoprobe shafts. However, insulation layers increase probe diameters, thereby increasing trauma to tissues probes traverse.

Thus, there is a widely recognized need for, and it would be highly advantageous to have, devices and methods enabling to deliver a plurality of cryoprobes to a treatment target absent the above-mentioned disadvantages.

### SUMMARY OF THE INVENTION

The present invention relates to use of an introducer for delivering thermal ablation probes to an organic target, and particularly for delivering multiple probes in a configuration and orientation enabling efficient and thorough ablation of a large target of complex shape. Preferred embodiments include introducers having individual probe channels shaped to direct inserted probes to diverge as they advance from the introducer into body tissues, probes designed and constructed to bend in selected manner when exiting the introducer, and probes and introducers comprising attachment mechanisms for fastening an introducer to an organic target during advancement of multiple probes from introducer to target.

The present invention successfully addresses the shortcomings of the presently known configurations by providing devices and methods enabling easy and rapid deliver of a plurality of treatment to a treatment target deep within a body, and further enabling to position treatment heads of the probes in a designed configuration appropriate for treating a large treatment target of complex shape, while yet requiring only a single incision to deliver such tools to a target and to provide feedback information as to the tool's deployment and position. The present invention further successfully addresses the shortcomings of the presently known configurations by providing devices and methods enabling to use a first of said plurality of probes to immobilize a target organ with respect to that first probe during insertion of others of that plurality of treatment probes.

The present invention further successfully addresses the shortcomings of the presently known configurations by providing devices and methods enabling to deliver a plurality of cryoprobes to a treatment target deep within a body and further enabling to position treatment heads of the probes in a designed configuration appropriate for treating a large treatment target of complex shape, which devices are operable to protect healthy tissues from damage by contact with cold shafts of cooling cryoprobes.

According to one aspect of the present invention there is provided an C. apparatus for thermal treatment of an organic target within a body of a patient, comprising: an introducer operable to introduce at least one thermal treatment probe into the body and to deliver a distal portion of the probe to a vicinity of the target, the introducer having a distal exterior wall, a distal end, and a longitudinal axis and being characterized by a distance D1 defined as a maximum of radial distances between points on the distal exterior wall and the longitudinal axis; and at least one thermal treatment probe operable to be introduced into the body through the introducer, the cryoprobe having a distal end, the introducer and the at least one probe being so designed and configured that there exists a distance L1 such that if the probe is advanced through the introducer so that the distal end of the probe is advanced beyond the exterior wall of the introducer by at least distance L1, then a distance D2, defined as a radial distance between the distal end of the probe and a linear extension of the longitudinal axis of the introducer, will be greater than distance D1.

According to further features in preferred embodiments of the invention described below the introducer is further operable to introduce a plurality of thermal treatment probes into the body and to deliver distal portions of the plurality of probes to the vicinity of the target; and the apparatus is so designed and configured such that there exists a distance L2 such that if a plurality of flexible cryoprobes is advanced through the introducer so that distal tips of the plurality of probes extend beyond a distal end of the introducer by at least distance L2, then the distal tips form a dispersed configuration characterized in that a distance D3, defined as a maximum of distances of the distal tips one from another, is greater than double the distance D2.

According to further features in preferred embodiments of the invention described below the introducer further comprises a plurality of curved channels, each channel sized to accommodate a treatment probe.

According to still further features in preferred embodiments of the invention described below the curved channels diverge as they approach a distal end of the introducer. Preferably the apparatus comprises an axially located straight channel, the plurality of curved channels being positioned around the axially located straight channel.

The apparatus may further comprise a sharp distal end shaped to facilitate penetration of the introducer into body tissues.

According to a preferred embodiment at least one probe is a pre-bent probe having a distal portion which is operable to assume a straight configuration when constrained to do so, and which assumes a bent configuration when unconstrained.

Preferably, the apparatus further comprises a plurality of pre-bent probes each having a distal portion which is operable to assume a straight configuration when constrained to do so, and which assumes a bent configuration when unconstrained.

According to further features in preferred embodiments of the invention described below the introducer comprises a plurality of channels each sized to accommodate one of the plurality of probes.

According to further features in preferred embodiments of the invention described below the apparatus further comprises an attaching mechanism enabling to attach the introducer to tissue of the target.

The attaching mechanism may comprise a hook operable to penetrate tissue. Preferably the hook is of corkscrew-shaped spiral construction and may be attached to a distal portion of the introducer or to a distal portion of a probe operable to be advanced towards the target from within the introducer.

Alternatively, the attaching mechanism comprises a distal portion operable to be cooled to freezing temperatures while in contact with the target, thereby creating adhesion between the attaching mechanism and frozen tissues of the target.

Further alternatively, the apparatus further comprises a channel for applying suction to the target, thereby attaching a portion of the apparatus to the target.

According to further features in preferred embodiments of the invention described below the introducer comprises an echogenic portion, and/or at least one probe comprises an echogenic portion.

According to further features in preferred embodiments of the invention described below the at least one probe comprises a marking describing a characteristic of the probe, and a shaft of the at least one probe comprises markings serving to indicate position of the probe within the introducer when the probe is inserted in the introducer and a distal end of the probe is advanced to a position near a distal end of the introducer.

According to further features in preferred embodiments of the invention described below the apparatus further comprises a position sensor operable to report position of the probe within the introducer and an actuator operable to induce movement of the probe within the introducer.

The probe may be a Joule-Thomson cryoprobe, the apparatus then further comprising a source of high-pressure cooling gas, a source of high-pressure heating gas, and a controller operable to control deliver of the high-pressure gasses to the cryoprobe.

According to further features in preferred embodiments of the invention described below the apparatus further comprises a sensor interface unit operable to receive data from a sensor and to calculate and send commands to the controller.

Preferably, the interface unit is further operable to calculate an estimated future position of an ablation volume producible by thermal treatment probes introduced into the body through the introducer, the calculation being at least partially based on information received from the sensor.

Preferably, the interface unit is further operable to display the estimated ablation volume position, and is further operable to calculate a real-time estimate of an actual ablation volume position, the calculation being at least partially based on data received from the sensor.

Preferably, the interface unit is further operable to calculate in a first calculation an estimated future position of an ablation volume producible by thermal treatment probes introduced into the body through the introducer and to record the estimated future position, the first calculation being at least partially based on information received from a first sensor, and the interface unit is further operable to calculate in a second calculation a real-time estimate of an actual ablation volume position, the second calculation being at least partially based on data received from a second sensor. Preferably the interface unit is further operable to display a comparison of results of the first and second calculations.

According to further features in preferred embodiments of the invention described below the introducer comprises a channel sized to accommodate a treatment probe, the channel having a distal opening at a circumferential position on the introducer, the circumferential positioned being distanced from a distal end of the introducer.

Preferably, the apparatus further comprises a plurality of treatment probes operable to be inserted into the body through the introducer.

Preferably the introducer further comprises a sharp distal end shaped to facilitate penetration of the introducer into body tissues, and at least one of the plurality of cryoprobes comprises a sharp distal end operable to be positioned so as to be substantially flush with the sharp distal end of the introducer.

According to another aspect of the present invention there is provided an introducer operable to deliver a plurality of cryoprobes to a treatment target within a body of a patient, comprising a plurality of channels each sized to accommodate a cryoprobe, at least one of the channels being curved.

According to further features in preferred embodiments of the invention described below the introducer further comprises a straight central channel and a plurality of curved channels positioned around the central channel, and at least one of the curved channels curves away from the central channel as it approaches a distal end of the introducer.

Preferably, the curved channels are disposed symmetrically around the central channel, and distal ends of at least some of the plurality of channels diverge as they approach a distal end of the introducer.

Preferably, the introducer further comprises a position sensor for sensing a position of a treatment probe within the introducer, and an insertion device operable to advance a treatment probe within the introducer.

According to yet another aspect of the present invention there is provided an apparatus operable to deliver a plurality of cryoprobes to a target, comprising a body having a longitudinal axis; a plurality of channels within the body, each channel sized to accommodate a treatment probe; and at least one pre-bent probe operable to exit a distal portion of the introducer and to advance in a direction which is at an angle to the longitudinal axis of the introducer, and preferably comprising a plurality of pre-bent probes. The pre-bent probe may comprises stainless steel and may comprise shape memory metal.

According to further features in preferred embodiments of the invention described below, the apparatus further comprises a plurality of pre-bent treatment probes positioned within the plurality of working channels in such orientation that when the pre-bent treatment probes extend from a distal end of the introducer, a distance of one of the treatment heads from at least one other of the treatment heads is greater than a diameter of the introducer.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1a is a simplified schematic of a cryoprobe introducer, according to an embodiment of the present invention;

FIG. 1b(i) is a simplified schematic of a distal end of the introducer of FIG. 1a, according to an embodiment of the present invention;

FIG. 1b(ii) is a simplified schematic of a distal end of the introducer of FIG. 1a, showing an alternate configuration, according to an embodiment of the present invention;

FIGS. 1c, 1d, and 1e are simplified schematics showing successive stages of a method using an introducer and a plurality of treatment probes to treat a surgical target, according to an embodiment of the present invention;

FIG. 1f is a simplified schematic of a probe having a distal portion formed as a corkscrew, according to an embodiment of the present invention;

FIGS. 1g and 1h are two simplified views of a treatment probe introducer comprising an attachment mechanism for attaching the introducer to a treatment target, according to an embodiment of the present invention;

FIGS. 2a-2d are simplified schematics of a pre-bent treatment probe and an introducer for inserting that pre-bent probe into a treatment target, according to an embodiment of the present invention;

FIG. 2e is a simplified schematic of an introducer operable to introduce a plurality of pre-bent probes into a treatment target, according to an embodiment of the present invention;

FIG. 2f is a simplified schematic showing the introducer of FIG. 2e with probes advanced therethrough and operated in cryoablation, according to an embodiment of the present invention;

FIG. 2g is a simplified schematic showing a pre-bent probe with a long arc of curvature, according to an embodiment of the present invention;

FIG. 3a is a simplified schematic of an introducer 320 having a sharp distal end, according to an embodiment of the present invention;

FIG. 3b is a simplified schematic of an alternative configuration of an introducer having a sharp distal end, according to an embodiment of the present invention;

FIG. 4 is a simplified schematic of an introducer for pre-bent probes having a sharp distal end, according to an embodiment of the present invention;

FIGS. 5a(i), 5a(ii) and 5b are simplified schematics of components of an apparatus for insertion of pre-bent treatment probes into a body, according to an embodiment of the present invention; and

FIGS. 6a and 6b are simplified schematics of systems for cryoablation comprising multi-probe introducers, according to embodiments of the present invention.

### DESCRIPTION OF THE PREFERRED EMBODIMENTS

The present invention relates to devices and methods for thermal ablation of a surgical target within a body of a patient. Specifically, the present invention can be used to deliver a plurality of thermal ablation probes such as cryoprobes to an organic target, the probes being delivered in a configuration and orientation enabling efficient and thorough ablation of a large target of complex shape.

Preferred embodiments presented hereinbelow include a thermal probe introducer designed to penetrate into a body and formed to accommodate a plurality of thermal probes, the introducer comprising individual probe channels each sized to accommodate a thermal probe which may be advanced therethrough, the channels being shaped to direct probes inserted therethrough to diverge upon exiting the introducer. Additional preferred embodiments include probes designed and constructed to bend in selected manner when exiting a distal portion of an introducer, and introducer/probe apparatus comprising attachment mechanisms for attaching an introducer and/or a first probe to a treatment target, thereby facilitating accurate delivery of multiple probes to that target through an introducer.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

To enhance clarity of the following descriptions, the following terms and phrases will first be defined:

The phrase “heat-exchanging configuration” is used herein to refer to component configurations traditionally known as “heat exchangers”, namely configurations of components situated in such a manner as to facilitate the passage of heat from one component to another. Examples of “heat-exchanging configurations” of components include a porous matrix used to facilitate heat exchange between components, a structure integrating a tunnel within a porous matrix, a structure including a coiled conduit within a porous matrix, a structure including a first conduit coiled around a second conduit, a structure including one conduit within another conduit, or any similar structure.

The phrase “Joule-Thomson heat exchanger” as used herein refers, in general, to any device used for cryogenic cooling or for heating, in which a gas is passed from a first region of the device, wherein it is held under higher pressure, to a second region of the device, wherein it is enabled to expand to lower pressure. A Joule-Thomson heat exchanger may be a simple conduit, or it may include an orifice, referred to herein as a “Joule-Thomson orifice”, through which gas passes from the first, higher pressure, region of the device to the second, lower pressure, region of the device. A Joule-Thomson heat exchanger may further include a heat-exchanging configuration, for example a heat-exchanging configuration used to cool gasses within a first region of the device, prior to their expansion into a second region of the device.

The phrase “cooling gasses” is used herein to refer to gasses which have the property of becoming colder when passed through a Joule-Thomson heat exchanger. As is well known in the art, when gasses such as argon, nitrogen, air, krypton, CO₂, CF₄, and xenon, and various other gasses pass from a region of higher pressure to a region of lower pressure in a Joule-Thomson heat exchanger, these gasses cool and may to some extent liquefy, creating a cryogenic pool of liquefied gas. This process cools the Joule-Thomson heat exchanger itself, and also cools any thermally conductive materials in contact therewith. A gas having the property of becoming colder when passing through a Joule-Thomson heat exchanger is referred to as a “cooling gas” in the following.

The phrase “heating gasses” is used herein to refer to gasses which have the property of becoming hotter when passed through a Joule-Thomson heat exchanger. Helium is an example of a gas having this property. When helium passes from a region of higher pressure to a region of lower pressure, it is heated as a result. Thus, passing helium through a Joule-Thomson heat exchanger has the effect of causing the helium to heat, thereby heating the Joule-Thomson heat exchanger itself and also heating any thermally conductive materials in contact therewith. Helium and other gasses having this property are referred to as “heating gasses” in the following.

As used herein, a “Joule Thomson cooler” is a Joule Thomson heat exchanger used for cooling. As used herein, a “Joule Thomson heater” is a Joule Thomson heat exchanger used for heating.

The terms “ablation temperature” and “cryoablation temperature”, as used herein, relate to the temperature at which cell functionality and structure are destroyed by cooling. According to current practice temperatures below approximately −40° C. are generally considered to be ablation temperatures.

The term “ablation volume”, as used herein, is the volume of tissue which has been cooled to ablation temperatures by one or more cryoprobes.

As used herein, the term “high-pressure” as applied to a gas is used to refer to gas pressures appropriate for Joule-Thomson cooling of cryoprobes. In the case of argon gas, for example, “high-pressure” argon is typically between 3000 psi and 4500 psi, though somewhat higher and lower pressures may sometimes be used.

The terms “thermal ablation system” and “thermal ablation apparatus”, as used herein, refer to any apparatus or system useable to ablate body tissues either by cooling those tissues or by heating those tissues.

For exemplary purposes, the present invention is principally described in the following with reference to an exemplary context, namely that of cryoablation of a treatment target by use of cryoprobes operable to cool tissues to cryoablation temperatures. It is to be understood that invention is not limited to that exemplary context. The invention is, in general, relevant to thermal treatment of any surgical target by means of one or more treatment probes delivered to that target through an introducer. For simplicity of exposition, cryoprobes are presented in the Figures and reference is made to cryoprobes hereinbelow, yet all such references are to be understood to be exemplary and not limiting. Thus, discussion of cryoprobes hereinbelow may be understood to apply also to thermal probes of other sorts. Similarly, references to cryoablation of tissues are also to be understood as exemplary and not limiting. Thus, references to cryoablation are to be understood as referring also to non-cryogenic thermal ablation, and to non-ablative cryogenic treatment of tissues.

It is expected that during the life of this patent many relevant cryoprobes and cryoprobe introducers will be developed, and the scope of the terms “cryoprobe” and “introducer” is intended to include all such new technologies a priori.

As used herein the term “about” refers to ±10%.

In discussion of the various figures described hereinbelow, like numbers refer to like parts. The drawings are generally not to scale. Some optional parts may be drawn using dashed lines.

For clarity, non-essential elements are omitted from some of the drawings. Some optional elements or optional configurations appear drawn with dashed lines in the figures.

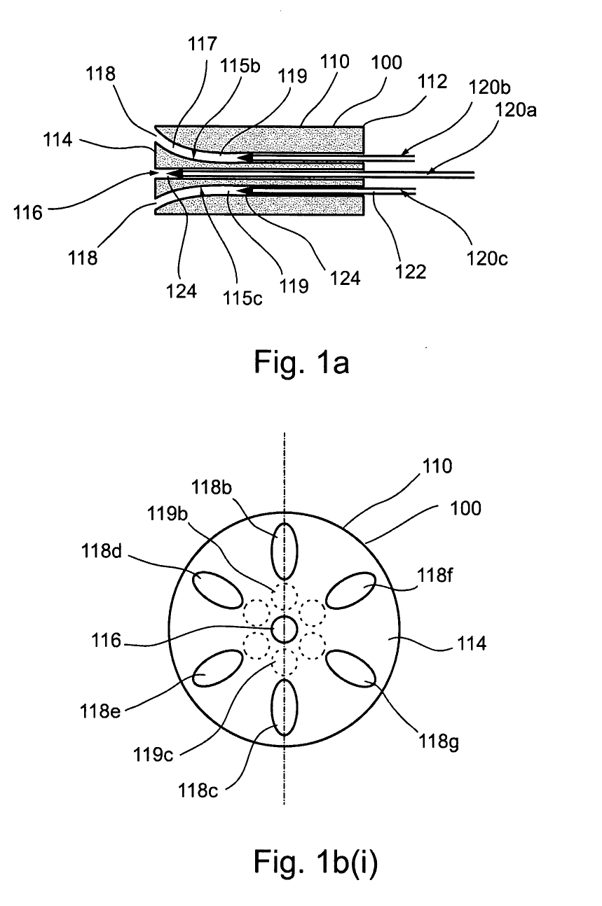

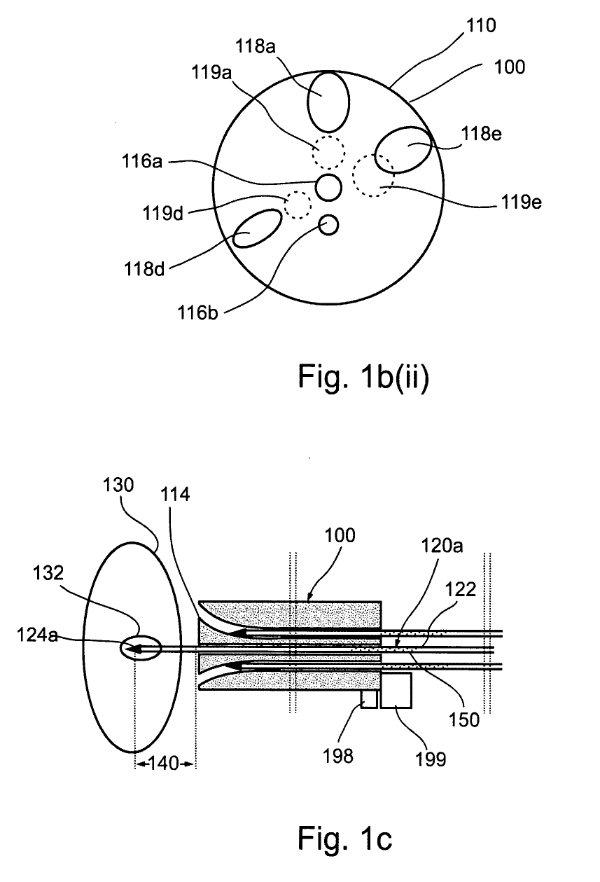

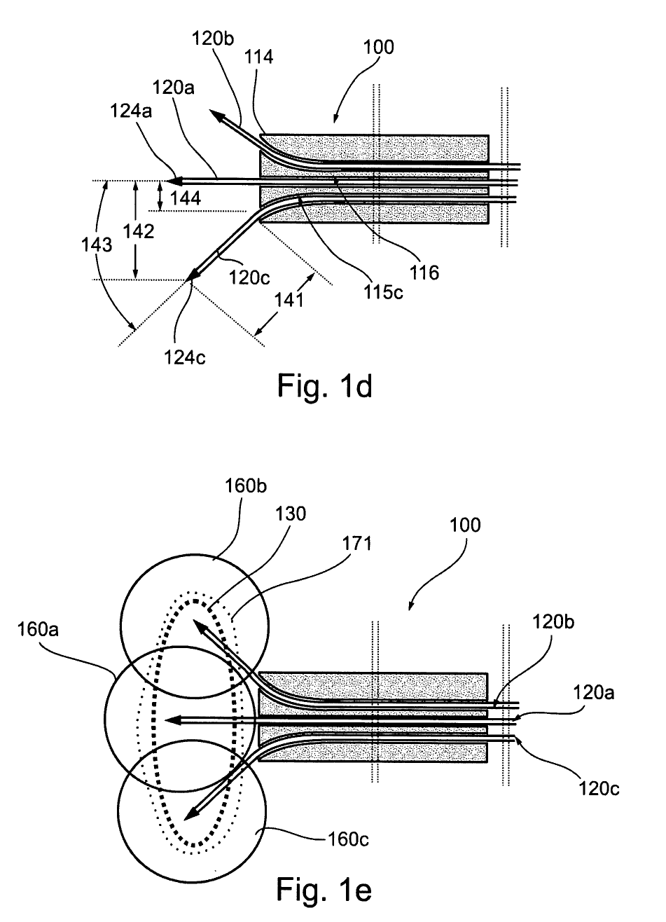

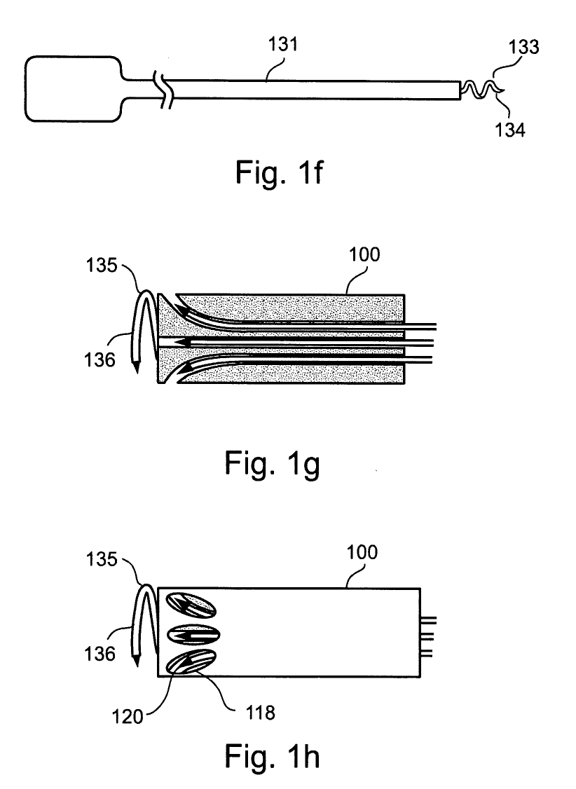

Attention is now drawn to FIG. 1a, which is a simplified schematic of a cryoprobe introducer, according to an embodiment of the present invention.

FIG. 1a presents an axial cross-sectional view of introducer 100. Introducer 100 comprises an elongated body member 110 (shortened, for clarity, in the Figure) having a proximal portion 112 and a distal portion 114. Distal portion 114 may be sharpened to facilitate penetration of introducer 100 into and through body tissues, or may be rounded so as to avoid inadvertent damage to body tissues (as when e.g. introducer 100 is introduced into a body through a trocar), or may be flat as shown in FIG. 1a, or may have any other convenient shape.

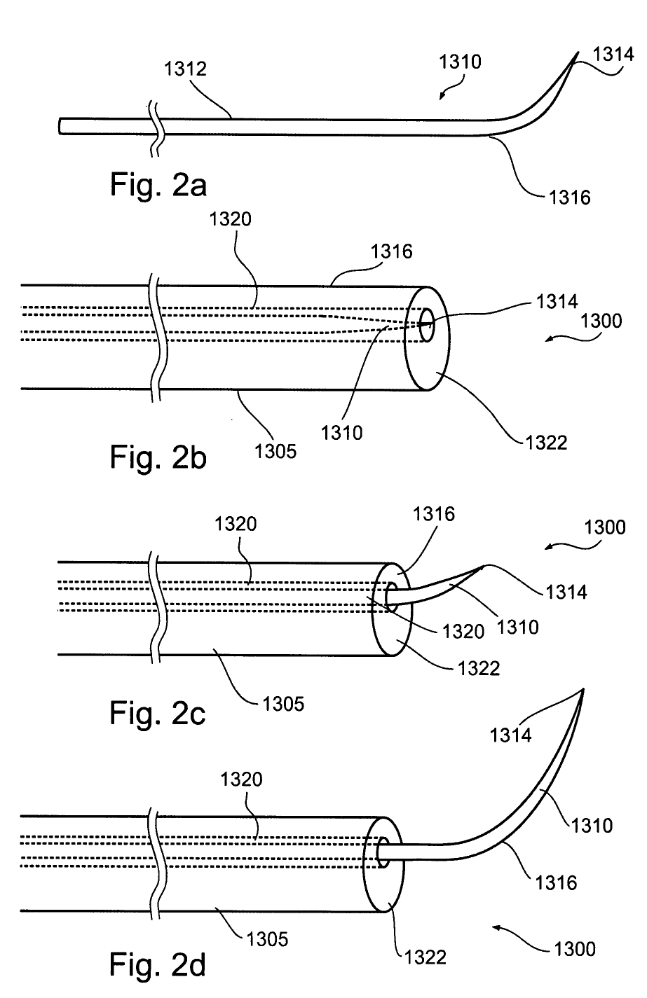

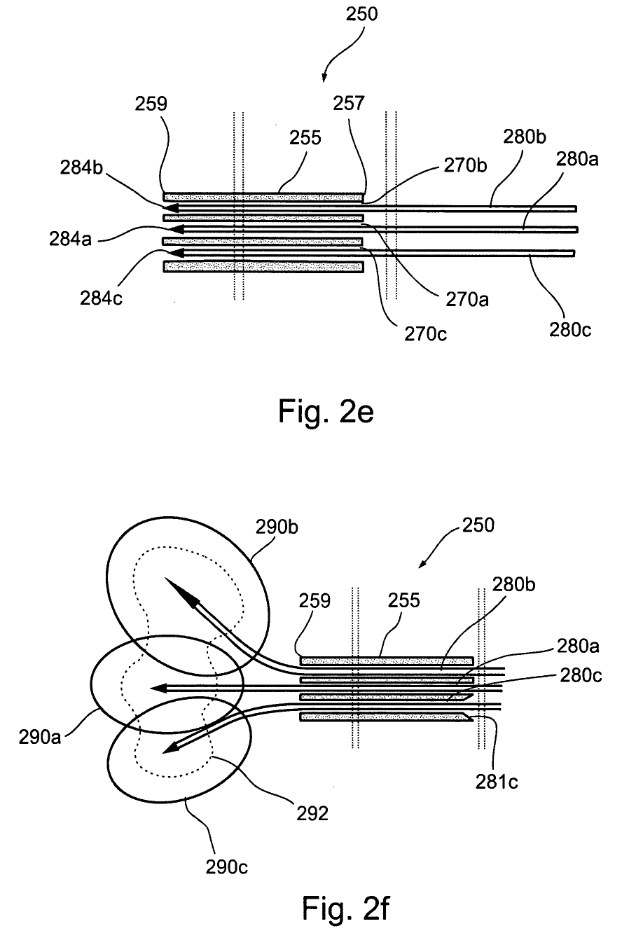

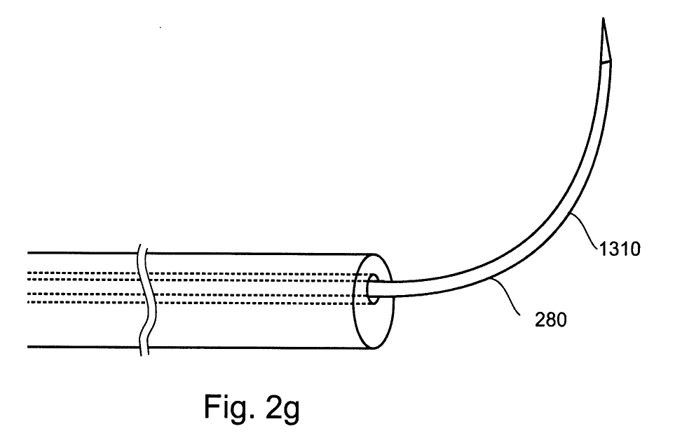

Introducer 100 comprises one or more curved channels 115, and optionally one and/or more straight channels 116 extending from proximal portion 112 to distal portion 114 of introducer 100. Each of channels 115 and 116 is sized to accommodate a cryoprobe or other surgical tool. Optionally, channels 115 and 116 may comprise an internal coating of low friction material such as Teflon, and/or may be lubricated, to facilitate insertion therethrough of cryoprobes or other tools. Proximal ends of channels 115 and 116 are preferably formed with a conical depression (such as is shown at 281c of FIG. 2f) to facilitate insertion of probes therein.

Introducer 100 is shown in FIG. 1a with cryoprobe 120a inserted in straight channel 116, and cryoprobes 120b and 120c inserted in curved channels 115b and 115c respectively. Each of curved channels 115 preferably comprises a straight section 119, a curve 117 and a distal opening 118. Each cryoprobe 120 comprises a cryo-tip 124 operable to cool tissue to cryoablation temperatures and optionally also operable to heat, and a shaft 122 operable to transport cryogen to and from cryo-tips 124. In a preferred embodiment, cryo-tips 124 are sharpened to facilitate penetration into target tissues. Shafts 122 are preferably semi-rigid, sufficiently strong to enable probes 120 to be pushed forward to advance through channels 115/116, yet sufficiently flexible to allow probes 120 to follow curves 117 of curved channels 115. Of course, the specific configuration presented in FIG. 1a is merely exemplary; multiple additional channels 115 and 116 may be provided, and tools other than cryoprobes 120 may be used therein.

In a preferred embodiment of the present invention a straight channel 116 is surrounded by a plurality of curved channels 115, each channel 115 curving outwards as shown in FIG. 1a (only channels 115b and 115c are shown in this cross section).

Attention is now drawn to FIG. 1b(i), which is a simplified schematic of a distal end of introducer 100, according to an embodiment of the present invention. In an exemplary embodiment of introducer 100 shown in FIG. 1b(i), six distal openings (labeled 118b-118g) of six curved channels 115 are shown to surround a distal opening of straight channel 116 on distal portion 114 of introducer 100. Distal openings 118b-118g are oval in appearance due to their angular orientation with respect to distal end 114 of introducer 100. Positions of straight sections 119 of curved channels 115 are shown as dashed circles in FIG. 1b(i).

It is noted that whereas the Figures here discussed present introducer 100 as straight (though containing curved channels), in an optional alternate configuration introducer 100 may also be curved, to suit the convenience of a surgeon in handling surgical tasks for which a curved introducer would be more convenient.

It is further noted that both introducer 100 and cryoprobes 120 may comprise echogenic marking patterns facilitating their observation by means of surgical visualization modalities such as ultrasound, MRI, or X-ray.

Attention is now drawn to FIG. 1b(ii), which is a simplified schematic of a distal end of introducer 100, showing an alternative configuration thereof, according to an embodiment of the present invention. The exemplary configuration of introducer 100 shown in FIG. 1b(ii) serves to demonstrate that the number of channels 115 and 116, their locations, their sizes, and the degree of curvature provided by various channels 115 may all vary to suit specific surgical tasks or indeed to suit preferences of various surgeons. FIG. 1b(ii) presents a front view of distal portion 114 of introducer 100, showing a plurality of non-symmetrically disposed, unequally sized and unequally curved channels. Specifically, two straight channels and three curved channels are seen. Straight channels 116a and 116b differ in size. Channel 116b, smaller in size than channel 116a, may for example be used for delivering a thermal sensing probe comprising one or more thermal sensors. Three curved channels 115 are represented by their openings 118a, 118d and 118e, and by their straight portions (shown as dotted circles) 119a, 119d and 119e respectively. Comparison of sizes and positions of the various elements shows that the channels 115 represented in FIG. 1b(ii) include a variety of sizes, positions, and curvatures. Larger diameter channels having openings 118a and 118e, for example, might be used to deliver relatively large-diameter cryoprobes to a treatment target. Such larger-diameter channels might be used, for example, to deliver to a particular portion of a treatment target a large diameter probe having a larger-than-usual heat-removing capacity.

It is to be understood that the specific configurations presented by the Figures are exemplary and not limiting. In a recommended mode of practice, a large variety of sizes and shapes and configurations of introducers would be made available to a surgical practitioner, enabling him to select an introducer and set of treatment probes appropriate for each particular surgical task. In particular, the circular outer perimeter of introducer 100 as shown in FIG. 1b, and that of other introducers discussed herein, is not to be understood as limiting. Introducer 100 and other introducers here presented may have other cross sectional shapes than those shown in the drawings. For example, an oval shape is recommend for certain applications. Introducers may also have varying cross sectional shapes and varying dimensions along their lengths.

Introducers may also optionally comprise additional features not specifically presented in the figures. For example, an introducer may optionally comprise usable to introduce or remove fluids from the vicinity of the treated organ, such as channels provided to enable flushing an operating arena with saline to facilitate viewing or to prevent freezing of nearby tissue. Similarly, channels may be provided to enable passage of carbon dioxide used for inflation, suction used to remove blood, etc. Optionally, additional surgical instruments may be introduced through channels of an introducer, such as light sources, viewing instruments, ultrasonic instruments, etc.

Attention is now drawn to FIGS. 1c, 1d, and 1e, which are simplified schematics showing successive stages of a method for introducing a plurality of treatment probes into a treatment target and for ablating that target, according to an embodiment of the present invention.

Attention is first drawn to FIG. 1c, which presents a step of stabilizing an introducer with respect to a treatment target. The pair of dotted lines near the center of introducer 100 in FIG. 1c and in the following figures serves to indicate that although introducer 100 is here presented in shortened format for simplicity of the Figure, introducer 100 will in fact typically be relatively long and thin.

In a recommended mode of practice, introducer 100 is inserted into a natural or man-made cavity in a patient's body. Alternatively, introducer 100 may be provided with a sharp distal portion 114, and simply be inserted by force through body tissues until a target locus is reached. In either case, a surgeon directs introducer 100 to a treatment target 130, preferably utilizing an imaging modality such as ultrasound, x-ray, CT, or MRI to verify position of introducer 100. A surgeon may also utilize navigational technology such as motion sensors or position sensors, or may position introducer 100 under direct (e.g. endoscopic) visual guidance.

Cryoprobes 120 may be pre-loaded into introducer 100. Alternatively, introducer 100 may be introduced into the body and made to approach target 130, and probes 120 may then be inserted into the body through introducer 100 as needed.

Once introducer 100 is correctly positioned with respect to target 130, the surgeon inserts one of cryoprobes 120 into target 130. In most cases, insertion of a centrally positioned probe (probe 120a in FIG. 1c) will be appropriate.

In an optional but recommended step, the inserted probe 120 (120a in FIG. 1c) is then caused to be fastened to target 130. In a preferred embodiment, probe 120a is made fast to target 130 by being briefly activated in cooling. That is, cryogen flow is initiated, causing probe 120a to cool sufficiently to freeze adjacent tissue. This freezing process a small iceball 132 and causes tissue of the iceball to adhere to probe 120a, thereby firmly attaching probe 120a to target 130 at a fixed position. For this purpose probe 120a is operated for a short time or at a low cooling setting (e.g. using low gas pressure in a Joule-Thomson probe, or simply repeatedly turning on and off supply of cryogen to probe 120a.)

In an alternative embodiment, probe 120a may be provided with a distal end shaped as a corkscrew 134, as shown in FIG. 1f. In this case probe 120a is made fast to target 130 by the simple expedient of rotating probe 120a while gently pressing it forward, much as a corkscrew is rotated to cause it to enter and make fast to a cork in a bottle of wine. In a further alternative embodiment, suction is applied through one or more of channels 115/116, which channel(s) may be empty or may be loaded with a probe, causing adhesion of introducer 100 to target 130 while one or more treatment probes are inserted. Optionally, a dedicated suction channel (not shown) may be provided in introducer 100. In a further alternative embodiment, presented in detail in FIGS. 1g and 1h, introducer 100 may itself be provided with an attachment mechanism such as a distal freezing portion operable to stabilize position of target 130 with respect to introducer 100 during insertion of treatment probes through introducer 100 and into target 130, or a corkscrew 136 or other hooking device operable to stabilize position of target 130 with respect to introducer 100 during insertion of treatment probes through introducer 100 and into target 130.

Both before and after fixing probe 120a to target 130, distance 140 between distal end 114 of introducer 100 and tip 124a of probe 120a may be assessed using imaging modalities as mentioned above. Alternatively, distance 140 may be assessed by reading a scale 150 provided on shaft 122 of probe 120a. Scale 150 is designed to show distance of insertion of probe 120a into introducer 100, and may be designed to provide a direct readout of distance 140 by which probe 120a has advanced beyond distal end 114 of introducer 100.

In an alternative embodiment, introducer 100 comprises a position sensor 198 (e.g. an electronic position sensing device) operable to measure and report position of probe 120a (and/or other probes) with respect to introducer 100. Sensor 198 may be an optical sensor operable to read scale 150, or any other type of position or movement sensor.

In a further alternative embodiment, introducer 100 comprises a mechanical or electro-mechanical insertion device 199 operable to advance probe 120a and/or other probes 120 within introducer 100. Preferably, sensor 198 and actuator 199 are combined into a common device operable to individually advance and retract a plurality of probes 120 according to commands of a surgeon or under algorithmic control, to desired distances or positions. Depending on clinical considerations, probes may be advanced individually, in groups, or all together. For example, a surgeon might wish to advance a central probe individually, and then advance a set of peripheral probes collectively. Optionally, probes intended to advance and retract in unison may be mechanically joined.

Attention is now drawn to FIG. 1d, which shows a stage in a preferred mode of operation whereby, once probe 120a (or another probe of inserter 100, or inserter 100 itself) is successfully anchored to target 130, a surgeon advances additional probes through introducer 100 and into target 130.

As may be seen from FIG. 1d, a distance 142 between tip 124c of probe 120c and tip 124a of probe 120a may be calculated by knowing distance 141 by which probe 120c is advanced beyond distal face 114 of carrier 100, angle 143 of curved channel 115c as it exits introducer 100, and distance 144 between curved channel 115c and strait channel 116.

Additional probes are preferably advanced one after another. Alternatively, all the additional probes may be advanced at once. Distances and relative positions of all probe tips one with respect to another may be calculated as shown in FIG. 1d.

It is noted that after a first probe (e.g. 120a) is fixed to target 130, movement of introducer 100 is still possible by rotation of introducer 100 around shaft 122 of that first probe, and by sliding introducer 100 along the shaft of the fixed probe, thereby changing the distance 140 from introducer 100 to target 130. Once a second probe is fixed to target 130, however, rotational motion of introducer 100 with respect to target 130 is largely inhibited.

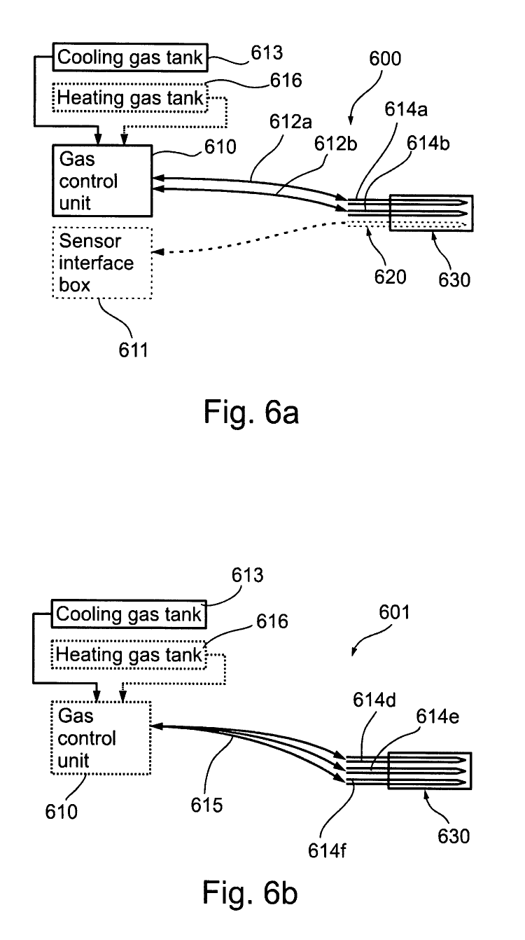

Attention is now drawn to FIG. 1e, which presents a third stage in use of introducer 100 and probes 120, according to a preferred embodiment of the present invention. Once a plurality of probes 120 have been inserted into target 130, a surgeon initiates a thermal ablation cycle. In a preferred embodiment of the present invention probes 120 are Joule-Thomson cryoprobes, and initiation of a thermal ablation cycle comprises enabling flow of cooling gas into expansion chambers within probes 120. FIG. 1e shows probes 120a, 120b and 120c creating iceballs 160a, 160b, and 160c respectively. An isotherm 171 denoting a border of a volume of certain full tissue ablation may be calculated based on theoretical considerations, feedback from visualization modalities, and information from thermal sensors. Thermal sensors may be mounted inside or outside probes 120, may be mounted inside or outside introducer 100, may be mounted as independent probes and passed through a channel of introducer 100 into a vicinity of target 130, and/or may be introduced into a vicinity of target 130 from another source. A sensor interface unit 611 (shown in FIG. 6a) preferably comprises computing means for computing the above-mentioned calculations and display means for displaying the calculated values in graphic format.

In a preferred mode of operation, sensor interface unit 611 calculates an estimated ablation volume in advance of the thermal ablation cycle, based on known characteristics of probes 120 and of target 130 and known information regarding inserted positions of probes 120, and presents that calculated ablation volume (preferably graphically) to a surgeon, who then optionally adjusts positions of probes 120 until he is satisfied with the calculated ablation volume estimate. In a further preferred mode of operation, sensor interface unit 611 compares that calculated estimated ablation volume to a real-time estimate of an actual ablation volume during and after an ablation cycle, as a guide to the surgeon for when determining duration and intensity of cooling and as a guide to post-cooling evaluation of the ablation cycle. Additionally, of course, a temperature-sensitive imaging modality such as MRI may be used to monitor treatment progress, and/or ultrasonic viewing may be used to monitor size and position of iceballs created by the freezing process.

Attention is now drawn to FIG. 1f, which presents a simplified schematic of a treatment probe 131 having a hooking arrangement 133, preferably formed as a corkscrew 134, useable to secure introducer 100 to a treatment target 130, according to an embodiment of the present invention. Use of treatment probe 131 has been described hereinabove. It is noted that probe 131 may be a thermal treatment probe such as a probe 120, or a pre-bent probe 1310 as described hereinbelow. Alternatively, probe 131 may be used for anchoring only, and not be used for thermal treatment. In this case, after all thermal treatment probes have been introduced into the target, anchoring probe 131 may be replaced by a thermal treatment probe.

Attention is also drawn to FIGS. 1g and 1h, simplified views of an embodiment of introducer 100 which comprises an attaching mechanism 135 formed in this exemplary embodiment as a spiral hook 136 (i.e. a short spiral corkscrew shape), enabling to attach introducer 100 to a treatment target, thereby stabilizing introducer 100 with respect to the a treatment target during insertion of probes 120 into that target.

Attention is now drawn to FIGS. 2a-2d, which present simplified schematics of a pre-bent treatment probe and an introducer for inserting that pre-bent probe into a treatment target, according to an embodiment of the present invention.

FIGS. 2a-2d present a probe delivery apparatus 1300 operable to deliver a pre-bent probe 1310 to a treatment target. Pre-bent probe 1310 may be any thermal treatment probe or other surgical tool in probe form. In a preferred embodiment, pre-bent probe 1310 is a cryoprobe, and most preferably a Joule-Thomson cryoprobe operable to cool by decompression of a high-pressure cooling gas. Alternatively, probe 1310 may be an evaporative cryoprobe. Probe delivery apparatus 1300 comprises pre-bent probe 1310 and an elongated member 1305 containing at least one delivery channel 1320 having a distal opening 1322.

A pre-bent thermal ablation probe 1310 having a semi-rigid shaft 1312, a thermal tip 1314 and a bend 1316 may be seen in FIG. 2a. Fabrication processes known in the art enable to fabricate thermal ablation probe 1310 (e.g. of stainless steel) with a desired bent shape and a desired degree of springiness, so as to be flexible yet tending to spring back to a pre-determined bent shape. Alternatively, pre-bent probe 1310 may be constructed of Nitanol® shape memory metal. For convenient surgical practice it will be preferable to make available to a surgeon a plurality of pre-bent probes 1310, configured with a variety of bending angles, bending radius, and lengths of arc, from which plurality of probes a surgeon can select the probe or probes most appropriate for a particular task at hand, thus enabling a surgeon to control lateral displacement of a probe tip by selecting a probe with desired curvature characteristics and by controlling insertion distance for that probe. In a preferred embodiment, information about direction, radius, and length of bend is printed on the shaft of each probe 1310, preferably on a proximal portion of the shaft so as to be visible to an operator when probe 1310 is inserted in channel 1320. Also, as described above for probes 120, insertion distance of probe 1320, or a distance 140 by which probe 1310 extends beyond opening 1322 of channel 1320 may be shown in a scale on a visible portion of shaft 1312 of probe 1310.

If probe 1310 is a cryoprobe, shaft 1312 is connectable by flexible hose to a cryogen control unit 1235 (not shown) operable to regulate supply of a cryogen to probe 1310.

Pre-bent probe 1310 is semi-rigid and can be straightened and inserted into delivery channel 1320 as seen in FIG. 2b. Shaft 1312 is sufficiently strong that pushing on its proximal end causes its distal end to extend from delivery channel 1320, thereby enabling insertion of (preferably) sharpened thermal tip 1314 into ablation target tissue. Sharp tip 1314 of insertable probe 1310 may be conical in shape, ending in a distal point. Alternatively, sharp tip 1314 may be chisel shaped, or shaped as a pyramided, or slanted in similarity to a hypodermic needle, or shaped in any manner which facilitates insertion of probe 1310 into tissue.

Optionally, pre-bent probe 1310 may be rotated inside delivery channel 1320, so that bend 1316 may be directed towards a desired direction. Thus, by rotating at least one of a plurality of probes 1310 before advancing that probe into a target, a non-symmetrical configuration of probes may be created, appropriately positioned in treated tissue to enable appropriate thermal treatment of a non-symmetrical lesion. To facilitate this process, a handle may be provided on a proximal end of pre-bent probe 1310, the handle positioned and/or marked in a manner which indicates direction of bend and optionally degree of bend of the probe's distal end. Such a handle may then be useful to an operator for rotating and/or advancing probe 1310.

As shown in FIGS. 2c and 2d, a selected length of a distal portion of probe 1310 may be extended beyond distal end 1322 of delivery channel 1320, resulting in a selected degree of curvature of the exposed distal portion of probe 1310. A distal portion of probe 1310 of selected length may be extended from delivery channel 1320 before sharpened thermal tip 1314 is introduced into a target, or alternatively delivery channel 1320 may be positioned directly on a target tissue, and then an arc of probe 1310 of selected length may be extended within that target tissue. It is anticipated that, for target tissues with tough consistency, a fibroid for example, positioning delivery channel 1320 contiguous to the target prior to advancing probe 1310 from channel 1320 will generally be found to be a preferable method for introducing sharpened thermal tip 1314 into such a target. Markings on the proximal shaft of probe 1310 may be provided, which markings serve to indicate what distal length of probe 1310 extends beyond distal opening 1322 of delivery channel 1320 at any given time.

Attention is now drawn to FIG. 2e, which is a simplified schematic of an introducer operable to introduce a plurality of pre-bent probes into a treatment target, according to an embodiment of the present invention. Introducer 250 is structurally and functionally similar to probe delivery apparatus 1300 presented in FIGS. 4a-4d and discussed hereinabove, and comprises a plurality of channels 270 which are similar to channel 1320 discussed above.

Thus, introducer 250 comprises an elongated body 255 having a proximal end 257 and distal end 259, and a plurality of channels 270, each operable to accommodate and guide insertion of either a straight cryoprobe or a pre-bent cryoprobe into a vicinity of an ablation target. In FIG. 2e three channels 270 may be seen, labeled 270a, 270b, and 270c, each containing a probe 280, labeled 280a, 280b, and 280c respectively. Introducer 250 and its channels 270 are preferably substantially straight, but alternatively both introducer 250 and/or one or more channels 270 may be curved, or flexible.

In a preferred mode of operation a centrally located probe 280a is not pre-bent and is preferably used as anchoring probe as described hereinabove with respect to probe 120a of FIG. 1c. Thus, introducer 250 is inserted into a natural or man-made cavity in a body, and directed toward an ablation target such as target 130. Imaging modalities such as ultrasound, x-ray, CT, and MRI, or navigational tools, or direct visual guidance may be used to position introducer 250 with respect to a target 130. Some or all probes 280 may be pre-loaded in introducer 250, or alternatively probes 280 may be inserted into introducer 250 as needed.

After positioning introducer 250 a correct location relative a target 130, a surgeon preferably inserts a central probe 280a into the target tissue and fixes it there, either by using a brief activation of a cooling mechanism of probe 280, or by utilizing a corkscrew distal portion (or other similar hook or fastener) of probe 280a, as discussed in detail hereinabove with respect to probe 120a of FIG. 1c and probe 131 of FIG. 1f.

Distance between distal end 259 of the carrier 250 and tip 284a of probe 280a may optionally be assessed using the imaging modality, additionally or alternatively, a scale imprinted on a probe shaft may be used, enabling use of techniques for assessing, reporting, and controlling probe positions presented hereinabove in particular with reference to elements 198 and 199 of FIG. 1c and sensor interface unit 611 of FIG. 6a.

Attention is now drawn to FIG. 2f, wherein additional probes 280 are shown advanced into target tissue and operated to form iceballs and an ablation volume, substantially as described hereinabove with respect to FIG. 1e. Thus, FIGS. 1a-1f and FIGS. 2a-2f present substantially similar methods of operation, differing in that cryoprobes advancing through introducer 100 of FIGS. 1a-1e are caused to diverge as they exit from introducer 100 by diverging channels within introducer 100, whereas probes 280 advancing through introducer 250 of FIGS. 2e-2f are pre-bent probes, and are caused to diverge as they exit from introducer 250 by virtue of their pre-bent characteristic, as described above. Thus, described above with respect to probes 120, probes 280 may be advanced simultaneously or one after another, their positions upon exiting introducer 250 may be calculated and reported, their calculated positions may form a basis for calculating and reporting an estimated ablation zone, that estimated ablation zone may be compared in real time to an actual ablation zone calculated based on theoretical calculations and/or reported or verified by sensor measurements and information derived from imaging modalities, etc. Iceballs 290a, 290b and 290c and a calculated ablation zone 292 are shown in FIG. 2f, in similarity to those shown in FIG. 1e.

Optionally, pre-bent probes 280 may be rotated within channels 270, thereby controlling the orientation of their curvature.

Optionally at least one of proximal openings of the channels 270, for example opening 281c, may be of conical shape, to facilitate insertion of a curved probe therein. Channels are optionally coated with low friction material such as Teflon, or are lubricated, to facilitate insertion of probes into channels and to facilitate advancing of probes therethrough.

Optionally, probes 280 may be equipped with thermal sensors operable to provide information usable for measuring size and position of cooled volumes. Probes equipped with multiple sensors, with or without a cooling mechanism, may be used.

A variety of probes 280 may be used, providing a surgeon with his choice of size of their cryo-tips and cooling capacities, of diameters, of degree of curvature and lengths and positions of curved portions. FIG. 2g, a simplified schematic showing a pre-bent probe with a long arc of curvature, according to an embodiment of the present invention, is provided to demonstrate that probes 280/1310 may provide continuous curvature over a considerable length, and may achieve total curvature of 90° or more.

Introducer 250 and pre-bent probes 280 present two advantages over introducer 100 having curved channels 115. A first advantage is that introducer 250 may be made thinner than introducer 100, if introducer 100 includes curved channels 115. Use of curved channels in introducer 115 requires a larger diameter to accommodate the curve of channels 115. Introducer 250, in contrast, can present probes 280 which exit introducer 250 with a sideways vector, yet introducer 250 can be relatively narrow, because only straight channels need be used, yet a sideways vector is imparted to pre-bent probes 280. It is further noted that introducer 250 can be made thinner still if a single inner lumen (not shown in the figure) is used to provide passage to a plurality of pre-bent probes, which plurality of probes will nevertheless extend from a distal end of introducer 250 in divergent directions, since their direction on exiting introducer 250 depends on their pre-bent characteristic and their orientation within introducer 250 rather than on some characteristic of the channel through which they are introduced into a body.

Thinness of introducer 250 is advantageous, in that it enables introducer 250 to pass through narrow natural openings (e.g. a partially dilated cervix) and/or to cause relatively less trauma when forced through resistant tissues.

A second advantage of pre-bent probes over curved channels lies in the fact that after an introducer is positioned with respect to a target, exit directions of probes passing through curved channels 115 is largely or completely fixed. In contrast, after introducer 250 is anchored with respect to a target, exit orientation of probes 280 may nevertheless be modified simply by rotating probes 280 within their channels (or within a common single channel version of introducer 250) prior to causing them to exit introducer 250 and enter target tissue.

A third advantage of pre-bent probes over curved channels lies in their enabling to select probes with different degrees of bending to achieve a desired combination of axial and lateral positioning of treatment tips, even after introducer 250 has been inserted into a body and even after one or more introduced probes have been fixed in position with respect to a target.

It is noted that characteristics and features presented herein in the context of discussions of introducer 100 and of introducer 250, and in discussions of various other introducers presented in the Figures and discussed herein, may be freely combined in a single introducer, and all such combinations are contemplated as embodiments of the present invention. Thus an introducer might, for example, contain both curved channels for guiding flexible non-pre-bent probes and straight or curved channels for guiding pre-bent probes, thereby enhancing probe divergence and/or providing for a highly adaptable tool set useful for varied specialized applications. Another example of an introducer combining characteristics of introducers 100 and 250 would be an introducer designed to function as introducer 250 and comprising an attachment mechanism such as that presented in FIGS. 1g and 1h.

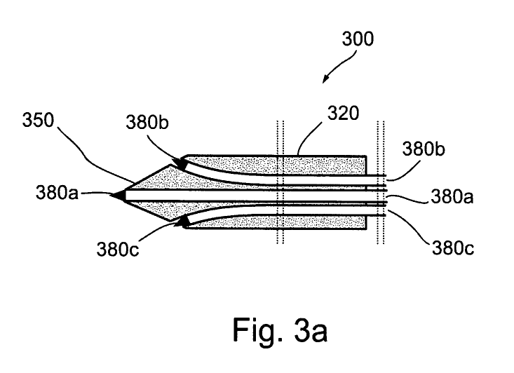

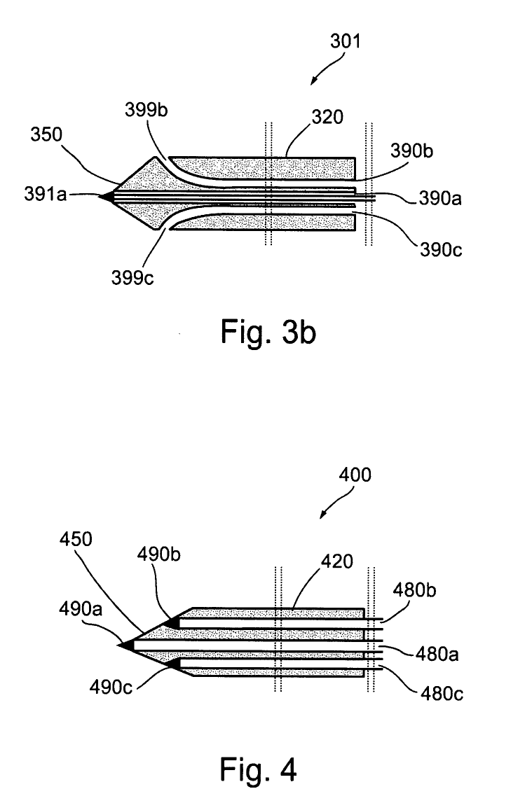

Attention is now directed to FIG. 3a, which is a simplified schematic of an introducer 300 having a sharp distal edge, according to an embodiment of the present invention. Introducer 300 has an elongated body 320 and is in most respects similar to introducer 100 described in detail hereinabove. Introducer 300 is distinguished in that distal edge 350 is sharp, and is designed to penetrate through tissues when inserted into a body.

Elongated body 320 comprises plurality of channels similar to channels 115 and 116 of FIG. 1a. Preferably, body 320 comprises at least one curved channel 115. In an exemplary embodiment shown in FIG. 3a, probe 380a is shown within a straight channel 116, and flexible probes 380b and 380c are shown inserted into curved channels 115 of introducer 300.

Preferably, operating tips of probes 380 are shaped to match the shape of sharp distal end 350 of introducer 300, so as to form a smoothly-cutting distal end 350 when probes 380 are inserted therein and positioned flush to distal face 350 of introducer 300.

To insert introducer 300 into a body, probes 380 are preferably positioned so that their distal faces are flush with distal face 350 of carrier 300, and are locked in that position. Introducer 300 is than forced into the body, cutting through tissue as required, thereby creating a man-made cavity in the body tissue. To this end, sharp distal end 350 of probe 300 may be shaped as a chisel, a cone, or any other shape having a sharp point and/or a sharp edge or edges.

Attention is now drawn to FIG. 3b, which is a simplified schematic of an alternative configuration of an introducer having a sharp distal end, according to an embodiment of the present invention.

FIG. 3b presents an introducer 301, similar to introducer 300 described above, yet different in that at least one of curved channels 115 (here labeled 390b and 390c) terminate in distal openings (399b and 399c) positioned on the circumference of shaft 320 of introducer 301, rather than on distal face 350 of introducer 301. These openings are thus not part of the distal cutting edge of introducer 301 and are not directly involved with cutting of tissue, therefore they do not require probes with matching shapes, as is preferred for probes supplied with introducer 300. Probes may also be inserted into introducer 301 after introducer 301 is inserted into a body, whereas probes of inserter 300 are preferably positioned within introducer 300 prior to insertion in a body, to distal channel openings interfering with smooth insertion and cutting by inserter 300.

Optionally, a straight central channel 390a is provided, and is fitted with a probe having a sharpened tip 391a configured to match the shape of sharpened distal end 350 of introducer 301, so that end 350 and probe tip 391a together form a distal shape sharp enough to cut and easily penetrate tissue. Alternatively, central channel 390a is absent and all channels have openings on the circumference of introducer body 320.

Attention is now drawn to FIG. 4, which is a simplified schematic of an introducer 400 having a sharp distal end, designed to deliver pre-bent probes to a treatment target, according to an embodiment of the present invention.

Carrier 400 comprises an elongated body 420 having a sharpened distal end 450.

Introducer 400 is similar to introducer 250, differing from it in that introducer 400 has a sharp distal end. In a preferred embodiment presented by FIG. 4, introducer 400 comprises an elongated body 420 which comprises a plurality of preferably substantially strait channels, through which probes 480 (only three: 480a, 480b and 480c are seen in this cross section) may be inserted. Preferably, central probe 480a is not pre-bent. Other probes 480 are preferably pre-bent and positioned to spread outwards when extended beyond distal end 450 of carrier 400.

Tips 490 of probes 480 (490a, 490b and 490c are seen in this cross section) are preferably shaped (as seen in FIG. 4) to match the shape of sharpened distal end 450, thereby creating a smooth surface which may effectively serve as a cutting edge when introducer 400 is used to forcefully penetrate tissue.

During insertion of introducer 400 into body tissue, probes 480 are preferably positioned flush with surface 450 as shown in the Figure, and locked in place within carrier 400, after which introducer 400 is forced into and through body tissue, creating a made cavity therein.

Sharpened distal end 450 may be shaped as a chisel, a cone, or any other shape having a sharp point and/or a sharp edge or edges.

In preferred embodiments, introducers 100, 250, 300, 301 and 400 comprise heat insulating material positioned to protect untreated tissue surrounding the introducers from low or high temperatures induced by operation of thermal probes contained within the introducers.

In preferred embodiments introducers 100, 250, 300, 301 and 400 comprise echogenic surfaces, preferably near their distal ends, which surfaces increase visibility of the introducers when those are used under ultrasonic guidance.

In preferred embodiments therapeutic probes for use with these introducers may comprise one or preferably multiple thermal sensors, and positions of those sensors may be marked by echogenic materials or by markings highly absorbent to x-rays, such marking serving to identify the locations of the thermal sensors to an operator.

According to another preferred embodiment, introducers 100, 250, 300, 301 and 400 may be equipped with a visual guidance system such as VT camera or fiber-optical scope, providing visual guidance during use of the introducers.

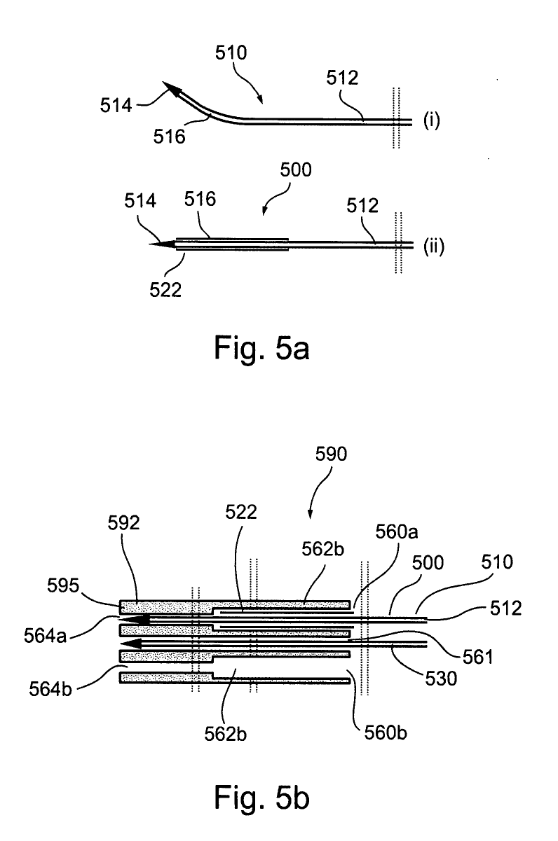

Attention is now drawn to FIGS. 5a(i), 5a(ii), and 5b, which are simplified schematics of components of an apparatus for insertion of pre-bent treatment probes into a body, according to an embodiment of the present invention.

FIG. 5a(i) presents a pre-bent probe 510 in isolation. Probe 510 comprises a semi-rigid shaft 512 having a bend 516, and a cryo-tip 514 at its distal end.

In FIG. 5a(ii) presents an assembly 500, comprising probe 510 and a sleeve 522 into which probe 510 may be inserted. When probe 510 is inserted into sleeve 522, bend 516 is straitened.

FIG. 5b presents an introducer 590 providing easy insertion of pre-curved probes. Carrier 590 comprises an elongated body 592 having distal end 595, and further comprises channels of two types, here labeled 560 and 561. In an exemplary embodiment presented in FIG. 5b, one channel 561 (preferably centrally positioned in carrier 590), and two channels 560 (labeled 560a and 560b) are seen in cross-section. It should be noted that the number of channel may be larger, and channel 561 may not be centrally located, and may be absent.

Central channel 561 has a same diameter throughout and is used with an un-bent probe 530.

In contrast, channels 560 each comprise a proximal section 562, of relatively larger diameter (labeled 562a and 562b in FIG. 5b) and a distal narrower section 564 of relatively narrower diameter (labeled 564a and 564b in the Figure).

When pre-bent probe assembly 500 is inserted into channel 560 (as depicted in channel 560a), sleeve 522 can enter into larger diameter section 562, but cannot advance into narrower section 564.

Thus, when an operator or mechanical mechanism pushes forward shaft 512, probe 510 exits introducer 590 and, no longer being constrained by sleeve 516, resumes its natural pre-bent curvature and follows a curved path into the body tissue.

Attention is now drawn to FIGS. 6a and 6b, which are simplified schematics of systems for cryoablation comprising multi-probe introducers, according to embodiments of the present invention.

FIG. 6a presents a system 600 which comprises a multiple-probe introducer 630. Introducer 630 may be any of the multiple-probe introducers discussed hereinabove. Two thermal treatment probes labeled 614a and 614b are shown in an exemplary embodiment presented by FIG. 6a, yet it is to be understood that additional probes may be used.

As shown in FIG. 6a, probes 614a and 614b are inserted in introducer 630 and each is separately connected (with hoses 612a and 612b respectively) to gas control unit 610. Gas control unit 610 controls supply of cooling gas from cooling gas tank 613 to probes 614.

Optionally, a heating gas tank 616 supplies heating gas used to heat probes 614, for example to thaw tissue adhering to probes 614 and thereby to facilitate their removal from treated tissue. Additionally or alternatively, heat may be supplied by electrical heater.

Optionally, one or more thermal sensing probes 620 may also be inserted in introducer 620 and thereby inserted into the body of a patient. Thermal sensing probes 620, if provided, preferably transmit data to a sensor interface unit 611, which may comprise means for recording thermal measurements, calculating thermal data, and reporting results of those calculations either graphically or otherwise. Sensor interface unit 611 is preferably a general-purpose calculation and control unit and may be operable to receive data from other sources, such as motion sensors, ultrasound or x-ray or other imaging modalities, pressure sensors, manual data input from a surgeon, etc. Interface unit 611 is also preferably operable to calculate control decisions under partial or total algorithmic control, and to send control commands to gas control unit 610 and to external units such as actuator 199 (shown in FIG. 1c).

In the embodiment shown in FIG. 6a, gas control unit 610 is operable to individually control gas flow to each of probes 614. Thus, probes 614a and 614b may be individually cooled or heated independently.

FIG. 6b presents a system 601, similar to system 600 in all respects except for the difference that probes 614 (here labeled 614d, 614e and 614f) connect to gas control unit 610 through a gas manifold 615 through which gas is supplied to probes 614 collectively. Thus, under control of gas control unit 610, probes 614 are all heated or all cooled in common.

It will be appreciated that while individual control of each probe 614 presents advantages of surgical flexibility, system 601 may be less expensive to build, easier to operate and more reliable. Optionally, systems 600 and 601 may be combined, with gas supply certain probes (e.g. central probes) be supplied individually and gas supply to other probes (e.g. peripheral probes) may be supplied collectively.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. An apparatus for thermal treatment of an organic target within a body of a patient, comprising:
(a) an introducer operable to introduce at least one thermal treatment probe into said body and to deliver a distal portion of said probe to a vicinity of said target, said introducer having a distal exterior wall, a distal end, and a longitudinal axis and being characterized by a distance D1 defined as a maximum of radial distances between points on said distal exterior wall and said longitudinal axis; and
(b) at least one thermal treatment probe operable to be introduced into said body through said introducer, said cryoprobe having a distal end,
said introducer and said at least one probe being so designed and configured that there exists a distance L1 such that if said probe is advanced through said introducer so that said distal end of said probe is advanced beyond said exterior wall of said introducer by at least distance L1, then a distance D2, defined as a radial distance between said distal end of said probe and a linear extension of said longitudinal axis of said introducer, will be greater than distance D1.

2. The apparatus of claim 1, wherein
(c) said introducer is further operable to introduce a plurality of thermal treatment probes into said body and to deliver distal portions of said plurality of probes to said vicinity of said target; and
(d) said apparatus is so designed and configured that there exists a distance L2 such that if a plurality of flexible cryoprobes is advanced through said introducer so that distal tips of said plurality of probes extend beyond a distal end of said introducer by at least distance L2, then said distal tips form a dispersed configuration characterized in that a distance D3, defined as a maximum of distances of said distal tips one from another, is greater than double said distanced D1.

3. The apparatus of claim 1, wherein said introducer further comprises a plurality of curved channels, each channel sized to accommodate a treatment probe.

4. The apparatus of claim 3, wherein said curved channels diverge as they approach a distal end of said introducer.

5. The apparatus of claim 4, further comprising an axially located straight channel, said plurality of curved channels being positioned around said axially located straight channel.

6. The apparatus of claim 1, wherein said introducer further comprises a sharp distal end shaped to facilitate penetration of said introducer into body tissues.

7. The apparatus of claim 1, wherein said at least one probe is a pre-bent probe having a distal portion which is operable to assume a straight configuration when constrained to do so, and which assumes a bent configuration when unconstrained.

8. The apparatus of claim 1, further comprising a plurality of pre-bent probes each having a distal portion which is operable to assume a straight configuration when constrained to do so, and which assumes a bent configuration when unconstrained.

9. The apparatus of claim 8, wherein said introducer comprises a plurality of channels each sized to accommodate one of said plurality of probes.

10. The apparatus of claim 1, further comprising an attaching mechanism enabling to attach said introducer to tissue of said target.

11. The apparatus of claim 10, wherein said attaching mechanism comprises a hook operable to penetrate tissue.

12. The apparatus of claim 11, wherein said hook is of corkscrew-shaped spiral construction.

13. The apparatus of claim 11, wherein said hook is attached to a distal portion of said introducer.

14. The apparatus of claim 11, wherein said hook is attached to a distal portion of a probe operable to be advanced towards said target from within said introducer.

15. The apparatus of claim 10, wherein said attaching mechanism comprises a distal portion operable to be cooled to freezing temperatures while in contact with said target, thereby creating adhesion between said attaching mechanism and frozen tissues of said target.

16. The apparatus of claim 10, further comprising a channel for applying suction to said target, thereby attaching a portion of said apparatus to said target.

17. The apparatus of claim 1, wherein said introducer comprises an echogenic portion.

18. The apparatus of claim 1, wherein said at least one probe comprises an echogenic portion.

19. The apparatus of claim 1, wherein said at least one probe comprises a marking describing a characteristic of said probe.

20. The apparatus of claim 1, wherein a shaft of said at least one probe comprises markings serving to indicate position of said probe within said introducer when said probe is inserted in said introducer and a distal end of said probe is advanced to a position near a distal end of said introducer.

21. The apparatus of claim 1, further comprising a position sensor operable to report position of said probe within said introducer.

22. The apparatus of claim 1, further comprising an actuator operable to induce movement of said probe within said introducer.

23. The apparatus of claim 1 wherein said probe is a Joule-Thomson cryoprobe and said apparatus further comprises a source of high-pressure cooling gas, a source of high-pressure heating gas, and a controller operable to control deliver of said high-pressure gasses to said cryoprobe.

24. The apparatus of claim 23, further comprising a sensor interface unit operable to receive data from a sensor and to calculate and send commands to said controller.

25. The apparatus of claim 24, wherein said interface unit is further operable to calculate an estimated future position of an ablation volume producible by thermal treatment probes introduced into said body through said introducer, said calculation being at least partially based on information received from said sensor.

26. The apparatus of claim 25, wherein said interface unit is further operable to display said estimated ablation volume position.

27. The apparatus of claim 26, wherein said interface unit is further operable to calculate a real-time estimate of an actual ablation volume position, said calculation being at least partially based on data received from said sensor.

28. The apparatus of claim 24, wherein said interface unit is further operable to calculate in a first calculation an estimated future position of an ablation volume producible by thermal treatment probes introduced into said body through said introducer and to record said estimated future position, said first calculation being at least partially based on information received from a first sensor, and said interface unit is further operable to calculate in a second calculation a real-time estimate of an actual ablation volume position, said second calculation being at least partially based on data received from a second sensor.

29. The apparatus of claim 28, wherein said interface unit is further operable to display a comparison of results of said first and second calculations.

30. The apparatus of claim 1, wherein said introducer comprises a channel sized to accommodate a treatment probe, said channel having a distal opening at a circumferential position on said introducer, said circumferential positioned being distanced from a distal end of said introducer.

31. The apparatus of claim 1, further comprising a plurality of treatment probes operable to be inserted into said body through said introducer.

32. The apparatus of claim 31, wherein said introducer further comprises a sharp distal end shaped to facilitate penetration of said introducer into body tissues, and at least one of said plurality of cryoprobes comprises a sharp distal end operable to be positioned so as to be substantially flush with said sharp distal end of said introducer.

33. An introducer operable to deliver a plurality of cryoprobes to a treatment target within a body of a patient, comprising a plurality of channels each sized to accommodate a cryoprobe, at least one of said channels being curved.

34. The introducer of claim 33, further comprising a straight central channel and a plurality of curved channels.

35. The introducer of claim 34, wherein said plurality of curved channels are positioned around said central channel and wherein at least one of said curved channels curves away from said central channel as it approaches a distal end of said introducer.

36. The introducer of claim 35, wherein said curved channels are disposed symmetrically around said central channel.

37. The introducer of claim 33, wherein distal ends of at least some of said plurality of channels diverge as they approach a distal end of said introducer.

38. The introducer of claim 33, further comprising a position sensor for sensing a position of a treatment probe within said introducer.

39. The introducer of claim 33, further comprising an insertion device operable to advance a treatment probe within said introducer.

40. The introducer of claim 38, further comprising an insertion device operable to advance a treatment probe within said introducer.

41. An apparatus operable to deliver a plurality of cryoprobes to a target, comprising
(a) a body having a longitudinal axis;
(b) a plurality of channels within said body, each channel sized to accommodate a treatment probe; and
(c) at least one pre-bent probe operable to exit a distal portion of said introducer and to advance in a direction which is at an angle to said longitudinal axis of said introducer.

42. The apparatus of claim 41, further comprising a plurality of pre-bent probes.

43. The apparatus of claim 41, wherein said pre-bent probe comprises stainless steel.

44. The apparatus of claim 41, wherein said pre-bent probe comprises shape memory metal.

45. The apparatus of claim 42, further comprising a plurality of pre-bent treatment probes positioned within said plurality of working channels in such orientation that when said pre-bent treatment probes extend from a distal end of said introducer, a distance of one of said treatment heads from at least one other of said treatment heads is greater than a diameter of said introducer.
